(19) United States
(12) Patent Application Publication
(10) Pub. No.: US 2015/0342696 A1
(43) Pub. Date: Dec. 3, 2015

(54) ILLUMINATION APPARATUS AND MEDICAL APPARATUS USING SAME

(71) Applicant: PANASONIC INTELLECTUAL PROPERTY MANAGEMENT CO., LTD., Osaka (JP)

(72) Inventors: Tohru HIMENO, Osaka (JP); Kenji MUKAI, Osaka (JP); Yoko MATSUBAYASHI, Osaka (JP); Naoko TAKEI, Osaka (JP)

(73) Assignee: PANASONIC INTELLECTUAL PROPERTY MANAGEMENT CO., LTD., Osaka (JP)

(21) Appl. No.: 14/718,716

(22) Filed: May 21, 2015

(30) Foreign Application Priority Data
May 30, 2014 (JP) 2014-112802

(51) Int. Cl. A61B 19/00; F21V 9/08

(52) U.S. Cl. CPC A61B 19/5202; F21V 9/08

(57) ABSTRACT

An illumination apparatus includes a light emitting unit configured to emit illumination light including a first light having a first peak wavelength of a first peak in a first wavelength range of 495 nm to 510 nm and a second light having a second peak wavelength of a second peak in a second wavelength range of 610 nm to 680 nm. In the illumination apparatus, an intensity of the second light at the second peak wavelength is higher than an intensity of the first light at the first peak wavelength.

### CROSS-REFERENCE TO RELATED APPLICATION

This application claims priority to Japanese Patent Application No. 2014-112802 filed on May 30, 2014, the entire contents of which are incorporated herein by reference.

### TECHNICAL FIELD

Embodiments disclosed herein relate to an illumination apparatus and a medical apparatus using the same which facilitate discrimination between human skin and veins.

### BACKGROUND ART

Conventionally, in medical facilities such as hospitals, an illumination apparatus has been used in obtaining a color difference for facilitating discrimination between arteries and veins during, e.g., surgery. The illumination apparatus emits light having a spectral component to increase contrast of biological tissues.

Recently, in the field of such medical illumination apparatus, a long-lifespan reliable light emitting diode (LED) is used as a light source (see, e.g., Japanese Patent No. 4452607). The LED has the advantage of emitting light with low power consumption and high efficiency. The illumination apparatus described in Japanese Patent No. 4452607 includes a light source capable of emitting white light and a light quantity adjusting means capable of independently adjusting a quantity of a green light component. Thus, it is possible to increase the contrast of biological tissues by decreasing the quantity of the light at a wavelength of 380 nm to780 nm, which is a visible light component.

In an examination room or hospital room of general medical facilities including clinics, etc., a relatively simple medical practice such as an intravenous injection is frequently performed. In this case, the illumination apparatus having high discrimination between the patient's skin and veins may be suitably used in such medical equipment. However, when the illumination apparatus described in Japanese Patent No. 4452607 is used as an illumination for an operating room, the discrimination between veins and arteries is high but the discrimination between skin and veins is not necessarily high. Further, in order to improve the discrimination between a plurality of biological tissues such as veins, arterial blood, liver and lung, the illumination apparatus includes a plurality of light sources such as blackbody radiation light, white LED, two-wavelength LED, and second two-wavelength LED, which makes the illumination apparatus larger in size. As a result, it becomes not suitable for a general illumination for an examination room or a hospital room.

### SUMMARY OF THE INVENTION

In view of the above, the disclosure provides an illumination apparatus capable of improving discrimination between human skin and veins with simple configuration, and a medical apparatus using the same.

In accordance with an aspect of the present invention, there is provided an illumination apparatus including a light emitting unit configured to emit illumination light including a first light having a first peak wavelength of a first peak in a first wavelength range of 495 nm to 510 nm and a second light having a second peak wavelength of a second peak in a second wavelength range of 610 nm to 680 nm, an intensity of the second light at the second peak wavelength being higher than an intensity of the first light at the first peak wavelength.

Preferably, the first peak wavelength ranges from 505 nm to 510 nm.

Preferably, the second peak wavelength ranges from 630 nm to 680 nm.

More preferably, a full width at half maximum of at least one of the first peak and the second peak is equal to or less than 50 nm.

Further, is preferred that a ratio of total radiant energy of illumination light in the first wavelength range and in the second wavelength range to radiant energy of illumination light in a wavelength range of 380 nm to 780 nm is equal to or greater than about 0.6.

More preferably, the ratio is equal to or greater than 0.8.

In the illumination apparatus, the light emitting unit may include one or more single wavelength solid state light emitting elements, each single wavelength solid state light emitting element emitting one of the first light and the second light.

Further, the illumination apparatus may further include a diffusion plate configured to diffuse and radiate the illumination light emitted from the light emitting unit.

In accordance with another aspect of the present invention, there is provided a medical apparatus including an illumination apparatus, wherein the illumination apparatus includes a light emitting unit configured to emit illumination light including a first light having a first peak wavelength of a first peak in a first wavelength range of 495 nm to 510 nm and a second light having a second peak wavelength of a second peak in a second wavelength range of 610 nm to 680 nm, and wherein an intensity of the second light at the second peak wavelength is higher than an intensity of the first light at the first peak wavelength.

With the above configuration, since a difference in spectral reflectance between the skin on the vein and the skin therearound is large in a wavelength range of 600 nm to 780 nm, it is possible to facilitate the discrimination between human skin and veins only by making the emission level of the light having the second peak wavelength in a wavelength range of 610 nm to 680 nm higher than the emission level of the light having the first peak wavelength in a wavelength range of 495 nm to 510 nm.

### BRIEF DESCRIPTION OF THE DRAWINGS

The figures depict one or more implementations in accordance with the present teaching, by way of example only, not by way of limitations. In the figures, like reference numerals refer to the same or similar elements.

FIG. 1 is a side view of an illumination apparatus and a medical apparatus using the same according to one embodiment of the present invention.

FIG. 2 is a side cross-sectional view of the illumination apparatus.

FIG. 3 is a schematic block diagram of the illumination apparatus.

FIG. 4 is a diagram showing an example of the spectrum of the illumination light emitted from the illumination apparatus.

FIG. 5A shows a side cross-sectional view of a first light emitting unit of the illumination apparatus, and FIG. 5B shows a side cross-sectional view of a second light emitting unit of the illumination apparatus.

FIG. 6 is a side cross-sectional view showing another configuration example of the light emitting unit of the illumination apparatus.

FIG. 7 shows the spectrums of illumination lights emitted from illumination apparatuses of Example, Comparative example 1 and Comparative example 2.

FIG. 8A shows an image diagram showing an appearance of skin and veins when using a general illumination apparatus, and FIG. 8B shows an image diagram showing an appearance of skin and veins when using the illumination apparatus of Example.

FIG. 9 is a diagram explaining combination patterns of two peak wavelengths of the illumination light emitted from the illumination apparatus according to the embodiment of the present invention.

FIG. 10 is a diagram showing a relationship between a color difference and a percentage of total radiant energy of wavelength ranges of the illumination light emitted from the illumination apparatus according to the embodiment of the present invention.

FIG. 11 is a diagram illustrating a preferred color temperature zone of the illumination light emitted from the illumination apparatus according to the embodiment of the present invention.

### DETAILED DESCRIPTION

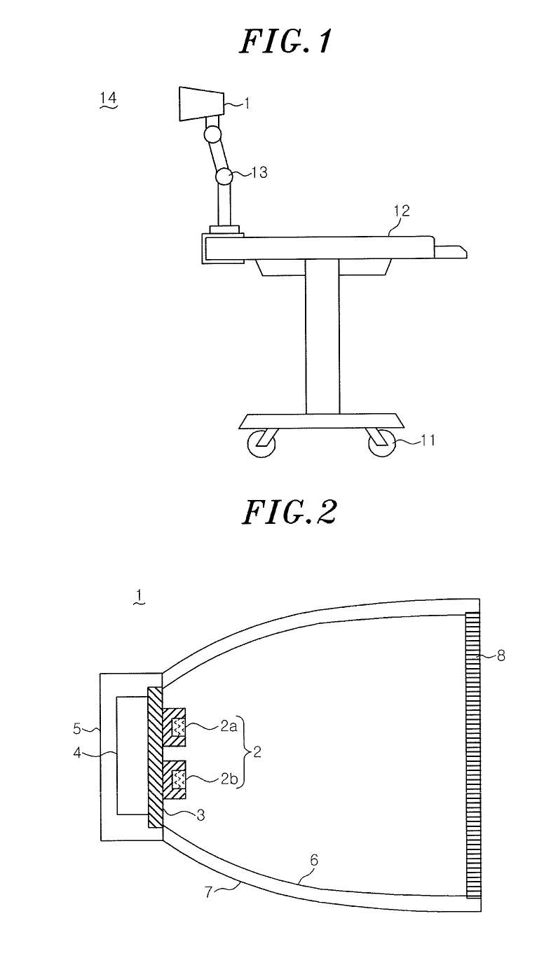

An illumination apparatus and a medical apparatus using the same according to an embodiment of the present invention will be described with reference to FIGS. 1 to 11. As shown in FIG. 1, an illumination apparatus 1 of this embodiment may be incorporated in a medical apparatus 14. In this case, the illumination apparatus 1 is installed in a nurse cart 12 having casters 11 through a movable arm 13. For example, the medical apparatus 14 is brought alongside the bed on which a patient required to have an intravenous injection lies, and a medical worker such as a nurse moves the movable arm 13 to an appropriate position at an appropriate angle such that light from the illumination apparatus 1 is irradiated to the patient's arm.

As shown in FIG. 2, the illumination apparatus 1 includes two types of light emitting units, i.e., a first light emitting unit 2a and a second light emitting unit 2b (collectively referred to as “light emitting unit 2”), a substrate 3 for mounting the light emitting unit 2 thereon, a power supply circuit 4 for supplying power to the light emitting unit 2, and an apparatus body 5 for accommodating the power supply circuit 4 therein while supporting the substrate 3. Further, the illumination apparatus 1 includes a reflection plate 6 for controlling distribution of illumination light emitted from the light emitting unit 2, a housing 7 which accommodates the reflection plate 6 and has an opening at the opposite side thereof from the substrate 3, and a diffusion plate 8 provided at the opening of the housing 7 to diffuse and radiate the illumination light emitted from the light emitting unit 2. In the apparatus body 5, a heat radiation plate (not shown) for radiating heat generated by the light emission of the light emitting unit 2 is provided.

In the illustrated example, the first and the second light emitting unit 2a and 2b are configured to be mounted on the substrate 3 by a surface-mount-device (SMD) method, but may be mounted by a chip-on-board (COB) method. In the COB method, instead of the diffusion plate 8, by adding a phosphor or a diffusing agent to sealing resin, it is possible to suppress the color unevenness or grainy feeling due to the light emission of the respective light emitting units 2a and 2b.

The reflection plate 6 is formed of a substantially bowl-shaped plate having a reflective property and is arranged to surround the periphery of the substrate 3. The reflection plate 6 may be provided as, for example, a light diffusing and reflecting plate which is fabricated by applying a highly reflective white paint to a resin structure having the bowl shape. Alternatively, instead of the reflection plate 6, a highly reflective white coating may be applied on the inner surface of the housing 7. The housing 7 may have a substantially bowl-shaped or tubular structure whose diameter is slightly larger than the reflection plate 6 to accommodate the reflection plate 6, and is formed of heat-resistant resin or a metal material such as aluminum.

The diffusion plate 8 is a plate-like member which is made of a milky white material obtained by adding diffusing particles such as titanium oxide to light-transmitting resin such as acrylic resin. Further, the diffusion plate 8 is machined to have substantially the same shape as the shape of the periphery of the opening of the housing 7. Alternatively, the diffusion plate 8 may be formed to have a rough surface by performing surface texturing or sandblast treatment on a front or a back surface of a transparent glass plate or a resin plate. With the diffusion plate 8, the lights emitted from the first and the second light emitting unit 2a and 2b are mixed with each other and it is possible to obtain a natural illumination light with less color unevenness and glare.

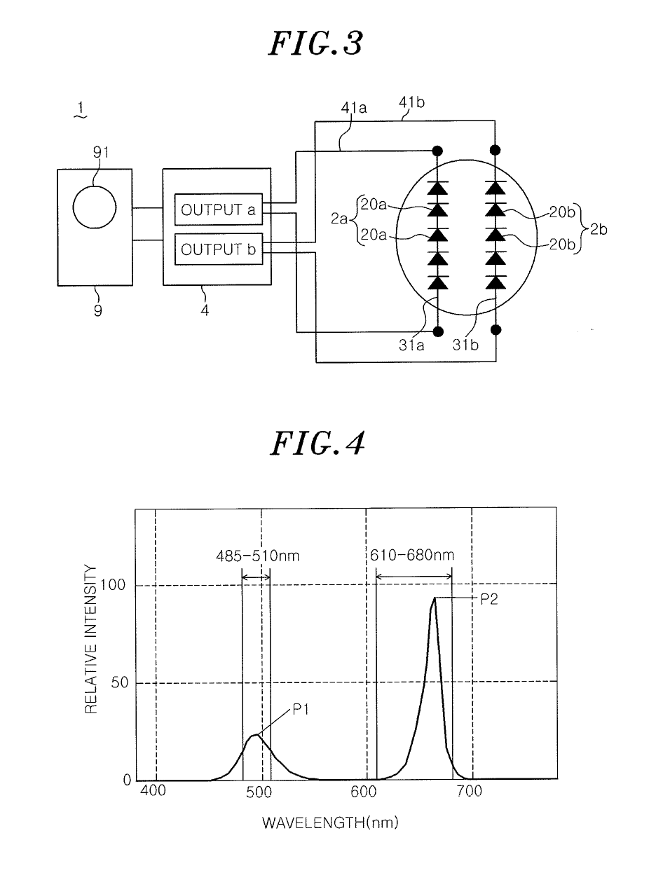

As shown in FIG. 3, each of the first and the second light emitting unit 2a and 2b includes a plurality of light emitting diodes (LEDs) 20a and 20b, respectively, and the plurality of LEDs 20a (20b) are mounted as a package on the substrate 3. The number of the LEDs 20a (20b) is not limited to the number in the illustrated example, and for example, the number of the LEDs 20a may be less than the number of the LEDs 20b. A wiring circuit (wiring circuits 31a and 31b in the illustrated example) is formed on the substrate 3 such that the same type of LEDs 20a (20b) are connected in series as a package. Further, electrode terminals of the wiring circuits 31a and 31b of the substrate 3 are respectively connected to output terminals a and b of the power supply circuit 4 through wirings 41a and 41b.

The substrate 3 is a substrate for a general-purpose light emitting module, and is made of metal oxide (including ceramic) such as aluminum oxide (Al₂O₃) having electrical insulation, metal nitride such as aluminum nitride (AlN), a material such as metal, resin, glass fiber or the like. A wiring circuit 31 formed on the substrate 3 is coated with an insulating material, and portions connected to positive and negative electrodes of the LEDs 20a and 20b and portions connected to the wirings 41a and 41b are exposed as respective electrode terminals (not shown).

The power supply circuit 4 serves as a power supply unit (not shown) for turning on and off the illumination apparatus 1, and includes a plurality of output terminals (outputs a and b in the illustrated example) corresponding to the types of the packages of the LEDs 20a and 20b. Further, the power supply circuit 4 has a rectifying and transforming circuit (not shown) which receives power from a commercial power source (not shown), and converts the power into a predetermined DC current, thereby controlling voltages applied to each of the LEDs 20a and 20b to correspond to a duty signal according to an emission level set by an operation unit 9.

The illumination apparatus 1 has the operation unit 9 (see FIG. 3, not shown in FIG. 2) for controlling the lighting and the emission level of the light emitting unit 2. The operation unit 9 may be provided in the apparatus body 5. Alternatively, the operation unit 9 may be provided at a position separated from the apparatus body 5 and configured to transmit a predetermined dimming control signal to the power supply circuit 4 wirelessly or in a wired manner. The operation unit 9 has a volume controller 91 such as a knob for adjusting the emission level of the light emitting unit 2, i.e., an intensity of the illumination light emitted from the light emitting unit 2.

As a user operates the volume controller 91 (the knob) to rotate, the illumination apparatus 1 may be turned on, and the emission level of the light emitting unit 2 may be changed according to a rotation range. The volume controller 91 may be configured such that light having a relatively low color temperature is irradiated while the emission level of the illumination apparatus 1 is relatively low, and a color temperature of the illumination light is gradually increased as the emission level is increased by further rotating the knob.

As shown in FIG. 4, the first light emitting unit 2a emits the light having a first peak wavelength in a wavelength range of 495 nm to 510 nm, and the second light emitting unit 2b emits the light having a second peak wavelength in a wavelength range of 610 nm to 680 nm. Further, in the illumination apparatus 1, the first light emitting unit 2a and the second light emitting unit 2b are controlled such that the emission intensity of the second peak wavelength is higher than the emission intensity of the first peak wavelength. The full width at half maximum of both or one of the first peak wavelength and the second peak wavelength is preferably 50 nm or less.

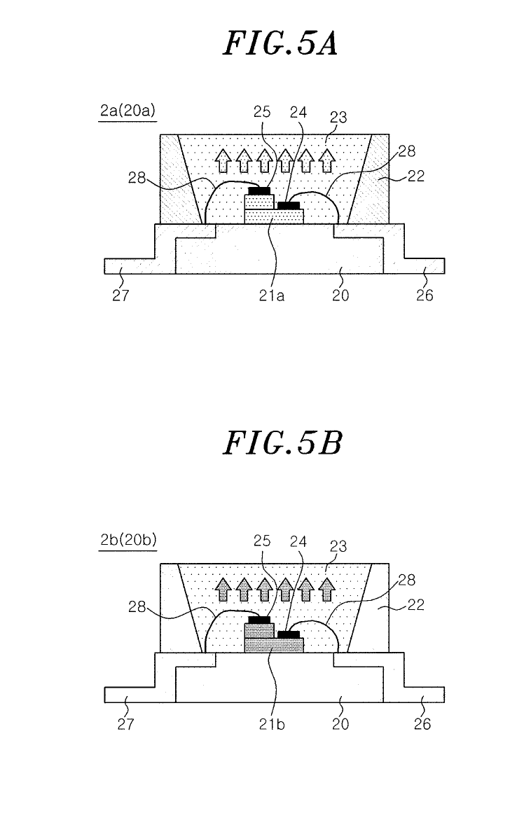

As shown in FIG. 5A, the LED 20a of the first light emitting unit 2a includes a base 20 having a substantially rectangular cross section, an LED chip 21a mounted on the base 20, a frame 22 having a recess to surround the LED chip 21a, and a filler 23 filled in the frame body 22. As the filler 23, silicon or the like is used. A cathode electrode 24 and an anode electrode 25 are provided on the LED chip 21a and are respectively connected to external connection electrodes 26 and 27 through wires 28. The inner peripheral surface of the frame 22 has a conical shape which opens in the irradiation direction of the light, and the inner peripheral surface of the frame 22 has a light reflecting function.

As the LED chip 21a, an element for emitting cyan (blue-green) light having a peak wavelength in a wavelength range of 495 nm to 510 nm, more preferably, a wavelength range of 505˜510 nm is used. In addition, a lens member (not shown) for controlling the distribution of the emitted light may be provided in the LED 20a.

As shown in FIG. 5B, the LED 20b of the second light emitting unit 2b has the same configuration as the LED 20a except that the LED chip 21b for emitting red light having a peak wavelength in a wavelength range of 610 nm to 680 nm, more preferably, a wavelength range of 630 nm to 680 nm is used.

It is preferable that at least one of the light having a first peak wavelength and the light having a second peak wavelength is obtained by a single wavelength solid state light emitting element (LED chip). When an illumination light is obtained by converting the light emitted from the LED chip using a phosphor, the spectrum of the illumination light includes an original peak wavelength of the light emitted from the LED chip itself. Thus, the emission intensity of a desired peak wavelength is not sufficiently obtained, and the full width at half maximum of the peak wavelength is easy to increase. Accordingly, there is a possibility that the contrast of the first peak wavelength and the second peak wavelength becomes blurred. On the contrary, by using bare solid state light emitting element without an additional component as an LED chip of one or both of the LEDs 20a and 20b, an unnecessary peak wavelength is reduced in the spectrum. As a result, it is possible to make the contrast of the first peak wavelength and the second peak wavelength clear.

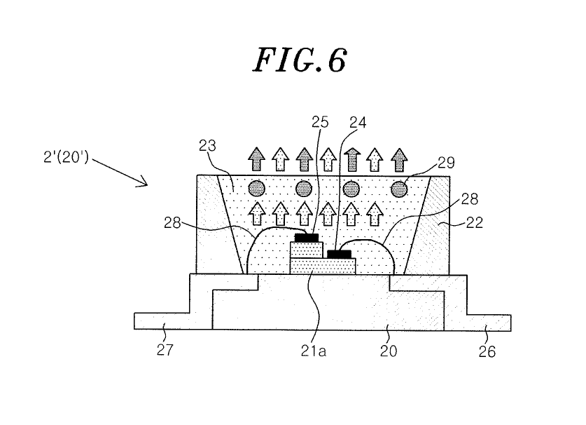

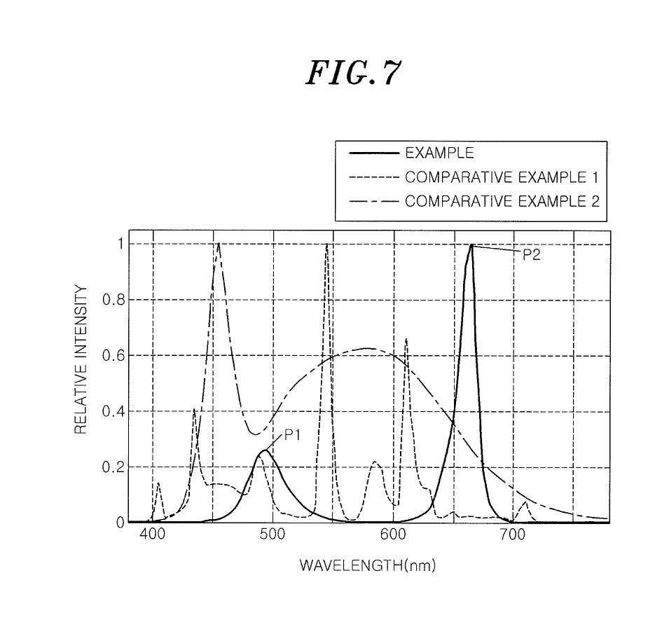

FIG. 6 shows a light emitting unit 2′ according to a modification of the embodiment. As shown in FIG. 6, the light emitting unit 2′ may be constituted by an LED 20′ in which a phosphor 29 converting the light emitted from the LED chip 21a into red light having a peak wavelength in a wavelength range of 610 nm to 680 nm is added to the filler 23. In this case, the light emitting unit 2′ may include a single type of light emitting unit, and the illumination light including two peak wavelengths can be emitted without requiring the diffusion plate 8.

Here, a test was performed on how the illumination apparatus 1 of the present embodiment can improve the discrimination between skin and veins compared to a general illumination apparatus. In the spectrum shown in FIG. 7, the solid line indicates the spectrum of the illumination light (Example (two-peak light)) of the illumination apparatus 1 of this embodiment, the dotted line represents the spectrum of the illumination light (Comparative example 1) of an illumination apparatus using a general three-wavelength fluorescent lamp, and the double-dotted line shows the spectrum of the illumination light (Comparative example 2) of a general indoor LED illumination apparatus.

The three-wavelength fluorescent lamp of Comparative example 1 is configured to emit the illumination light having a plurality peak wavelengths including peak wavelengths in R (red), G (green) and B (blue) wavelength ranges. The indoor LED illumination apparatus of Comparative example 2 emits the illumination light including the original peak wavelength of the light emitted from the blue LED and a gentle peak wavelength of light obtained by the wavelength conversion of the light emitted from the blue LED with a YAG-based yellow phosphor which is centered on the yellow wavelength.

Table 1 below shows optical characteristics (chromaticity coordinates (x, y), correlated color temperature Tcp [K], chromatic deviation duv from a black body radiation locus, and color rendering property (average color rendering index Ra)) of the illumination lights emitted from the respective illumination apparatuses of Example, Comparative example 1 and Comparative example 2.

TABLE 1

| | | | Tcp | | | |
|---|---|---|---|---|---|---|
| | x | y | [K] | duv | Ra | Remarks |
| Example | 0.3451 | 0.3516 | 4994 | −0.1 | −52 | Two peaks of |
| | | | | | | cyan and red |
| Comparative | 0.3451 | 0.3516 | 4994 | −0.1 | 84 | |
| example 1 | | | | | | |
| Comparative | 0.3434 | 0.3508 | 5057 | 0.3 | 86 | Blue LED + |
| example 2 | | | | | | YAG phosphor |

Also, Table 2 below shows color difference ΔE and color system coordinates L*, a*, b* in the skin on the veins and the skin around the veins by the illumination lights emitted from the respective illumination apparatuses of Example, Comparative example 1 and Comparative example 2.

TABLE 2

| | color | Skin | | | Skin | | |
|---|---|---|---|---|---|---|---|
| | difference | on the veins | | | around the veins | | |
| | ΔE | L* | a* | b* | L* | a* | b* |
| Example | 2.35 | 57.1 | 20.8 | 7.0 | 57.1 | 23.0 | 8.0 |
| Comparative | 1.25 | 55.0 | 6.8 | 11.1 | 54.9 | 7.8 | 12.0 |
| example 1 | | | | | | | |
| Comparative | 1.20 | 55.0 | 6.1 | 9.0 | 55.0 | 7.0 | 10.0 |
| example 2 | | | | | | | |

In case of Example, since the emission level of red light is high as compared to Comparative examples 1 and 2, a value of a* indicating a position near red between red and magenta in a CIELAB color space is high. On the other hand, since the emission level of cyan light is low as compared to red light, a value of b* indicating a position near yellow between yellow and blue is low.

The skin of human being (mostly white and yellow races) has a high difference in spectral reflectance between the skin on the veins and the skin around the veins in a wavelength range of 600 nm to 780 nm as compared with a wavelength range of 470 nm to 525 nm. Therefore, in Example, the emission level of red light having a peak wavelength in a wavelength range of 610 nm to 680 nm is increased, so the color difference ΔE between the skin on the veins and the skin around the veins becomes 2.35. Thus, it is possible to significantly improve the discrimination between the skin and the veins as compared to Comparative examples 1 and 2 (1.25 and 1.20, respectively).

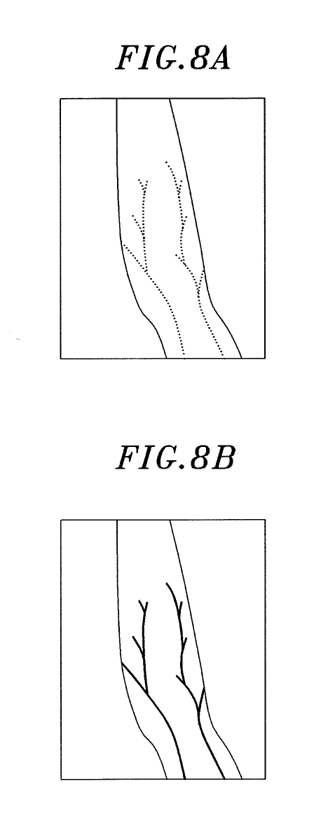

Also, in the case of using only the light emitting unit (second light emitting unit 2b) for emitting red light, the color of the skin looks like an unnatural color which is reddish. Therefore, by using the light emitting unit (first light emitting unit 2a) for emitting cyan light, it is possible to show the skin having a natural skin color by suppressing the redness of the skin while improving the discrimination between the skin and the veins. As a result, the veins shown in FIG. 8A can be easily distinguished as shown in FIG. 8B. Further, in the embodiment, as the light source, the light emitting unit that can emit illumination light including two peak wavelengths may be used and can be applied to a simple illumination apparatus rather than a large-scale apparatus such as a conventional illumination for an operating room.

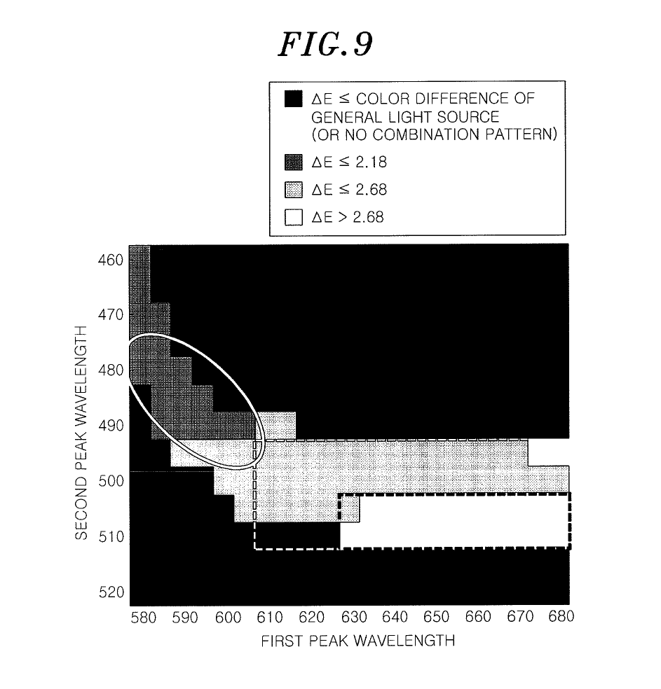

As shown in FIG. 9, the color difference ΔE between the skin on the veins and the skin around the veins changes depending on how to combine two peak wavelengths, i.e., a first peak wavelength and a second peak wavelength, of the illumination light emitted from the illumination apparatus 1. Regarding a pattern of a combination thereof, in the combination of 495 nm to 510 nm and 610 nm to 680 nm, the color difference ΔE becomes 2.18 or more, and in the combination of 505 nm to 510 nm and 630 nm to 680 nm, the color difference ΔE becomes 2.68 or more. In general, the color difference ΔE of 1.5 or more can be sensed by an average person, and if the color difference ΔE is 3.0 or more, anyone can sense a significant color difference. Further, in a combination of the region surrounded by an ellipse in FIG. 9, even if the color difference ΔE is 2.18 or less, there is a certain degree of discrimination, but color difference improvement is poor.

Therefore, the first peak wavelength is preferably present in a wavelength range of 495 nm to 510 nm, and more preferably present in a wavelength range of 505 nm to 510 nm. In order to increase the discrimination of the veins itself, as described above, it is necessary to increase the emission level (intensity) of the illumination light having a second peak wavelength in a wavelength range of 610 nm to 680 nm. On the other hand, in order to improve the discrimination between the skin on the veins and the skin around the veins, it is necessary to use the illumination light having a first peak wavelength present in a wavelength range of 495 nm to 510 nm, preferably, a wavelength range of 505 nm to 510 nm, at some emission level.

Particularly, the wavelength range of the first peak wavelength at which a high color difference ΔE is obtained is narrower than the wavelength range of the second peak wavelength at which a high color difference ΔE is obtained. Thus, as the light emitting unit (first light emitting unit 2a) for emitting light having a first peak wavelength, an LED (LED 20a) capable of adjusting the peak wavelength with high accuracy and reducing the full width (50 nm or less) at half maximum of the first peak wavelength is suitably used. Therefore, by using a single wavelength solid state light emitting element having a peak wavelength in a wavelength range of 505 nm to 510 nm as the LED chip 21a of the first light emitting unit 2a, it is possible to obtain a light emitting unit having desired emission characteristics.

The second peak wavelength preferably ranges from 610 nm to 680 nm, and more preferably 630 nm to 680 nm.

In the illumination apparatus 1, since the illumination light having two peak wavelengths of the first peak wavelength (495 nm to 510 nm) and the second peak wavelength (610 nm to 680 nm) is used, the color difference between the skin on the veins and the skin around the veins is large and light having a wavelength component other than the above wavelength range is desirably small.

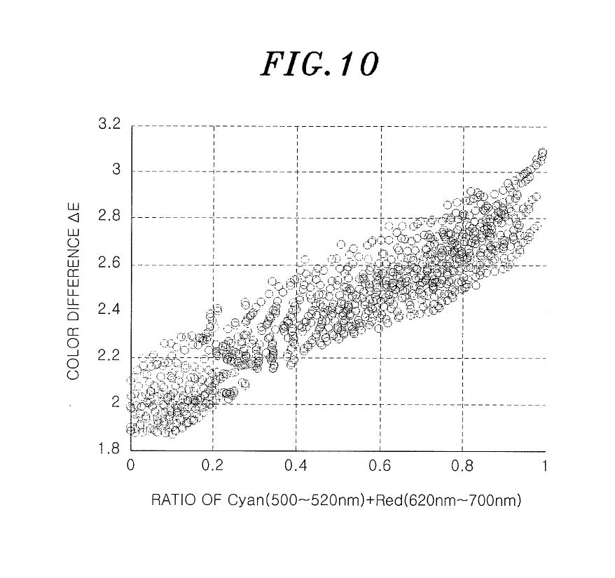

As shown in FIG. 10, a ratio of the total radiant energy of the illumination light in a wavelength range of 495 nm to 510 nm and a wavelength range of 610 nm to 680 nm to the radiant energy of the illumination light in a wavelength range of 380 nm to 780 nm corresponding to a wavelength zone of visible light has a strong positive correlation with the color difference ΔE. Specifically, a ratio of the total radiant energy of the illumination light in a wavelength range of 495 nm to 510 nm and a wavelength range of 610 nm to 680 nm to the radiant energy of the illumination light in a wavelength range of 380 nm to 780 nm is preferably 60% or more, and the ratio is more preferably 80% or more. That is, by increasing the emission level of the illumination light in the wavelength range of 495 nm to 510 nm and the wavelength range of 610 nm to 680 nm and decreasing the other wavelength range, the contrast of the two peak wavelengths increases, and the color difference ΔE can be larger.

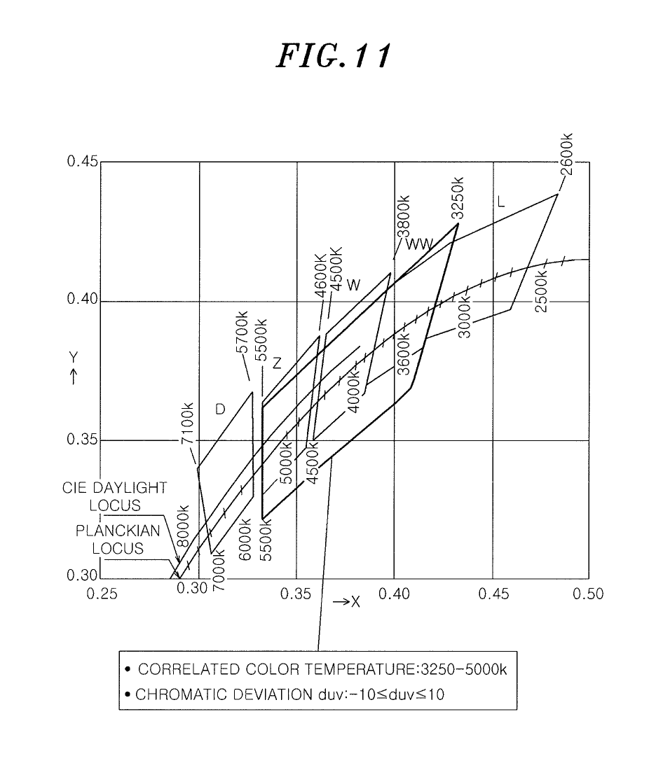

If the emission level of the second peak wavelength in a wavelength range of 610 nm to 680 nm is higher than the emission level of the first peak wavelength in a wavelength range of 495 nm to 510 nm, their emission ratio is not particularly limited, and the color temperature of the illumination light emitted from the illumination apparatus 1 is not limited. Further, the illumination light emitted from the illumination apparatus 1 preferably ranges from 3250 K to 5000 K of correlated color temperature including warm white, white and daylight white, among light source color classifications of LED standardized in, e.g., JIS Z 9112 as shown in FIG. 11. In this case, the chromaticity deviation duv is desirably in a range of −10≦duv≦10.

The present invention is not limited to the above-described embodiments and can be modified in various ways. For example, the illumination apparatus 1 may be provided in a medical hanger (not shown) suspended from the ceiling above the bed for a patient to supply medical gases or power without being limited to the medical apparatus 14 which is installed in a nurse cart as described above. Also, the illumination apparatus 1 may further include another light emitting unit for emitting light having wavelength characteristics other than that of the light emitting unit 2 as described above, and it may be used as a general illumination apparatus such as an interior lamp or a reading lamp. In this case, the another light emitting unit and the light emitting unit 2 may be selectively used through operation of a switch.

While the foregoing has described what are considered to be the best mode and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present teachings.

What is claimed is:

1. An illumination apparatus comprising:
a light emitting unit configured to emit illumination light including a first light having a first peak wavelength of a first peak in a first wavelength range of 495 nm to 510 nm and a second light having a second peak wavelength of a second peak in a second wavelength range of 610 nm to 680 nm,
wherein an intensity of the second light at the second peak wavelength is higher than an intensity of the first light at the first peak wavelength.

2. The illumination apparatus of claim 1, wherein the first peak wavelength ranges from 505 nm to 510 nm.

3. The illumination apparatus of claim 1, wherein the second peak wavelength ranges from 630 nm to 680 nm.

4. The illumination apparatus of claim 1, wherein a full width at half maximum of at least one of the first peak and the second peak is equal to or less than 50 nm.

5. The illumination apparatus of claim 1, wherein a ratio of total radiant energy of illumination light in the first wavelength range and in the second wavelength range to radiant energy of illumination light in a wavelength range of 380 nm to 780 nm is equal to or greater than about 0.6.

6. The illumination apparatus of claim 5, wherein the ratio is equal to or greater than 0.8.

7. The illumination apparatus of claim 1, wherein the light emitting unit includes one or more single wavelength solid state light emitting elements, each single wavelength solid state light emitting element emitting one of the first light and the second light.

8. The illumination apparatus of claim 1, further comprising a diffusion plate configured to diffuse and radiate the illumination light emitted from the light emitting unit.

9. A medical apparatus comprising an illumination apparatus,
wherein the illumination apparatus includes a light emitting unit configured to emit illumination light including a first light having a first peak wavelength of a first peak in a first wavelength range of 495 nm to 510 nm and a second light having a second peak wavelength of a second peak in a second wavelength range of 610 nm to 680 nm, and
wherein an intensity of the second light at the second peak wavelength is higher than an intensity of the first light at the first peak wavelength.
